(19) United States
(12) Patent Application Publication
(10) Pub. No.: US 2017/0333263 A1
(43) Pub. Date: Nov. 23, 2017

(54) ABSORBENT ARTICLE

(71) Applicant: OJI HOLDINGS CORPORATION, Tokyo (JP)

(72) Inventors: Izumi Tashiro, Tokyo (JP); Yoshihiro Takiyama, Tokyo (JP); Akira Sonoda, Tokyo (JP); Toshihiro Noda, Tokyo (JP); Daishi Nakayama, Tokyo (JP)

(21) Appl. No.: 15/534,207

(22) PCT Filed: Dec. 9, 2015
(86) PCT No.: PCT/JP2015/006135

§ 371 Date: Jun. 8, 2017

(30) Foreign Application Priority Data
Dec. 10, 2014 (JP) 2014-249965
Oct. 21, 2015 (JP) 2015-207417

(51) Int. Cl. A61F 13/513; A61F 13/511; A61F 13/49; A61F 13/539

(52) U.S. Cl. CPC A61F 13/513; A61F 13/539; A61F 13/5116; A61F 13/49001; A61F 2013/49092; A61F 2013/53908

(57) ABSTRACT

An absorbent article is provided, which is resistant to deformation and can maintain the breathability even under the force applied by various body movements, such as load of sitting. An absorbent article of the present invention includes a liquid permeable top surface sheet, a liquid impermeable back surface sheet, and an absorber disposed between the top surface sheet and the back surface sheet. Grooves extend on the top surface sheet side, and in each of the grooves, a plurality of recess portions formed by integrally joining the top surface sheet and the absorber are arranged. Each of the recess portions has a sidewall and a bottom.

### TECHNICAL FIELD

The present invention relates to an absorbent article, and particularly relates to an absorbent pad (urine pad) and a disposable diaper.

### BACKGROUND ART

In recent years, there has been an expanding demand for absorbent articles such as a disposable diaper (hereinafter also simply referred to as the “diaper”) using super absorbent polymers (hereinafter referred to as the “SAPs”) and non-woven fabric.

Various improvements have been made to an absorbent article such as a disposable diaper for better functions and wearing feeling thereof. An absorber used in the absorbent article mainly includes an absorbent core that is a mixture of fluff pulp and the SAPs and a core covering material such as tissue covering the absorbent core.

From the viewpoint of saving resources, reducing logistics cost, and improving storage efficiency at a retail store or the like for such absorbent articles, lightweight and compact absorbent articles called ultraslim absorbent articles or the like are demanded and becoming mainstream. Such ultraslim absorbent articles can be realized by reducing the weight and size of an absorber that accounts for a large part of the weight and volume of the absorbent article. More specifically, the absorbent core formed of a mixture of fluff pulp and the SAPs is reduced in thickness by increasing the relative SAP content or using a sheet-type SAP instead of the mixture.

In the absorbent article such as a disposable diaper, the absorber absorbs most of discharged body fluids. Here, if there is a gap between the absorbent article and the body, the body fluids may flow out and cause leakage. Therefore, the adhesion of the absorbent article is important as one of the functions required for the absorbent article. However, after the absorbent article absorbs body fluids, a space between the skin and the absorbent article gets highly damp, and gets poor in ventilation because the absorbent article sticks to the skin. Therefore, the absorbent article requires not only the adhesion but also the breathability. Particularly, the breathability of a diaper is required, because the diaper has a large area covering the entire lower part of the body, such as the abdomen, the buttocks, and the crotch portion, and also easily gets damp by absorbing a large amount of body fluids such as urine.

Patent Literature 1 describes an absorbent article that suppresses unpleasant dampness while ensuring absorptivity, in which a regular pattern having bumps and recesses alternately arranged therein is formed, such as a houndstooth-like pattern, on a top surface sheet that directly touches the skin in the absorbent article. Such an absorbent article causes only tips of the bump portion to touch the skin to reduce the contact area with the skin compared with those without any bump-recess pattern, and thereby suppresses unpleasant dampness and irritation.

### CITATION LIST

### Patent Literature

PTL 1: Japanese Patent Laid-Open No. 2003-275239

PTL 2: Japanese Patent Laid-Open No. 2015-16218

### SUMMARY OF INVENTION

However, the diaper covers a portion to which the pressure is applied by sitting, such as the buttocks and the crotch. Therefore, the pressure of sitting is often applied to the absorber and the top surface sheet.

The top surface sheet in the invention described in Patent Literature 1 is formed of non-woven fabric, and has bumps and recesses formed thereon by embossing the non-woven fabric. The top surface sheet itself is not very thick, and thus a difference in level between the bumps and recesses is also small. Therefore, the bump portions are partially collapsed under the load of sitting, resulting in a situation where the breathability effect by the embossing cannot be achieved. Particularly, the diaper covers a portion to which considerable load of sitting is applied, such as the buttocks and the crotch. Therefore, deformation under the pressure of sitting becomes a problem compared with a sanitary napkin.

It is a first object of the present invention to provide an absorbent article which is resistant to deformation even under the force applied by various body movements, such as load of sitting, and can maintain the breathability.

In the meantime, the flexibility of the absorber itself tends to be lost as the SAP content in the absorber is increased. Since a large twisting force is applied to the wearer's crotch region in the absorbent article by the movement of the wearer, particularly leg movements, such loss of the flexibility in the absorber leads to an increase in discomfort when he/she wears the absorbent article.

Therefore, for example, Patent Literature 1 proposes a technique in which compressed grooves are formed in an oblique lattice pattern on the surface of the absorber and the absorber is easily bent in the portion of the compressed grooves under the torsional force applied to the absorber. The configuration of the absorber described in Patent Literature 1 can maintain the flexibility of the absorber while improving the followability to the wearer. Furthermore, such an oblique lattice pattern is expected to improve diffusivity of liquids on the surface of the absorber, and to thus enhance the utilization efficiency of the absorber and realize cost reduction.

However, as for the absorber with the oblique lattice pattern as described in Patent Literature 1, pressing makes the entire absorber so stiff that the absorber is difficult to greatly twist. Therefore, although the absorber is twisted for various reasons when the absorbent article is worn, there is a problem that the absorber is flexible only in a certain direction since uniform arrangement of the lattice across the absorber determines the direction of the pattern.

Furthermore, there seems to be a problem that the formation of the oblique lattice pattern across the absorber reduces the breathability of the absorber in the extending direction.

The present invention is made in consideration of the above problems, and it is a second object of the present invention to provide an absorbent article which improves the flexibility of the absorber and thereby improves the breathability.

A first absorbent article of the present invention is an absorbent article including a liquid permeable top surface sheet, a liquid impermeable back surface sheet, and an absorber disposed between the top surface sheet and the back surface sheet, wherein grooves extend on a top surface sheet side of the absorbent article, and in each of the grooves, a plurality of recess portions formed by integrally joining the top surface sheet and the absorber are arranged, and each of the recess portions has a sidewall and a bottom wall.

In a second absorbent article of the present invention according to the first absorbent article of the present invention, the plurality of recess portions are arranged so as to form a plurality of first patterns extending in a direction intersecting with a direction in which two end regions of the absorber on both sides of a center region of the absorber face each other and form a plurality of second patterns extending in a direction intersecting with the first patterns.

In a third absorbent article of the present invention according to the first absorbent article of the present invention, the plurality of recess portions positioned in the center region of the absorber are arranged so as to form the first patterns and the second patterns, and the plurality of recess portions positioned in the two end regions on both sides of the center region of the absorber are arranged so as to form third patterns extending in the direction in which the two end regions facing each other.

The absorbent article of the present invention includes the ventilation grooves formed by integrally joining the top surface sheet and the absorber. Thus, the absorbent article of the present invention can maintain the breathability without the ventilation grooves being collapsed under the pressure of sitting.

Moreover, the present invention can provide an absorbent article which improves the flexibility of the absorber and improves the breathability.

### BRIEF DESCRIPTION OF DRAWINGS

FIG. 1 is a three-dimensional projection view showing an external appearance of an embodiment in which an absorbent article according to the present invention is applied to a pants-type disposable diaper;

FIG. 2 is a three-dimensional projection view seen from the back side, showing the lower part of a body wearing the diaper shown in FIG. 1;

FIG. 3 is a three-dimensional projection view showing the diaper shown in FIG. 1 in an unfolded and exploded state;

FIG. 4 is a partial top view seen from the top sheet side of the diaper shown in FIG. 1;

FIG. 5 is a cross-sectional view showing a crotch portion of the diaper taken along the line V-V in FIG. 4;

FIG. 6 is an enlarged view showing an example of a ventilation groove portion of the diaper shown in FIG. 1;

FIG. 7 is a cross-sectional view taken along the line VII-VII in FIG. 6;

FIG. 8 is a cross-sectional view taken along the line VIII-VIII in FIG. 6;

FIG. 9 is an enlarged view showing another example of the ventilation groove portion of the diaper shown in FIG. 1;

FIG. 10 is a cross-sectional view taken along the line X-X in FIG. 9;

FIG. 11 is a cross-sectional view taken along the line XI-XI in FIG. 9;

FIG. 12 is a partial top view showing a modified example of a ventilation groove pattern;

FIG. 13 is a partial top view showing a modified example of a ventilation groove pattern;

FIG. 14 is a partial top view showing a modified example of a ventilation groove pattern;

FIG. 15 is a perspective view showing a measurement device for measuring the air permeability of the diaper;

FIG. 16 is a partially ruptured plan view showing another embodiment seen from the skin contact surface side of the diaper shown in FIG. 1 in an unfolded state thereof;

FIG. 17 is a cross-sectional view showing the diaper taken along the line XVII-XVII in FIG. 16;

FIG. 18 is an exploded perspective view showing the diaper shown in FIG. 16 in an unfolded and exploded state;

FIG. 19 is a partial top view of an absorber portion of the diaper shown in FIG. 1 seen from the top sheet side according to Embodiment 2;

FIG. 20 is a partial plan view schematically showing an oblique lattice emboss pattern portion in FIG. 19;

FIG. 21 is a cross-sectional view showing the absorber portion taken along the line XXI-XXI in FIG. 19;

FIG. 22 is a schematic top view showing an example of an emboss pattern in Embodiment 3;

FIG. 23 is a schematic top view showing an example of an emboss pattern in Embodiment 4; and

FIG. 24 is a schematic top view showing an example of an emboss pattern in Embodiment 5.

### DESCRIPTION OF EMBODIMENTS

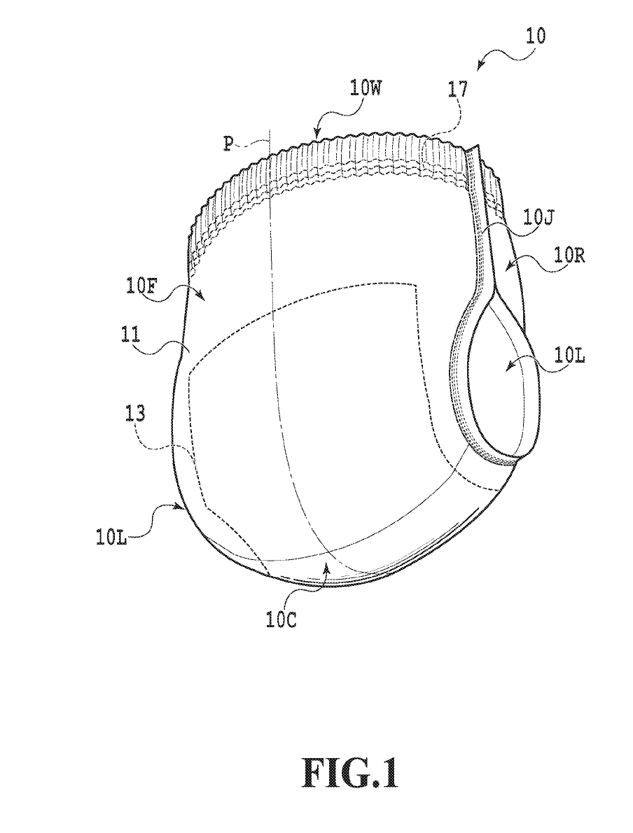

With reference to FIGS. 1 to 24, detailed description is given of embodiments of an absorbent article according to the present invention.

### Embodiment 1

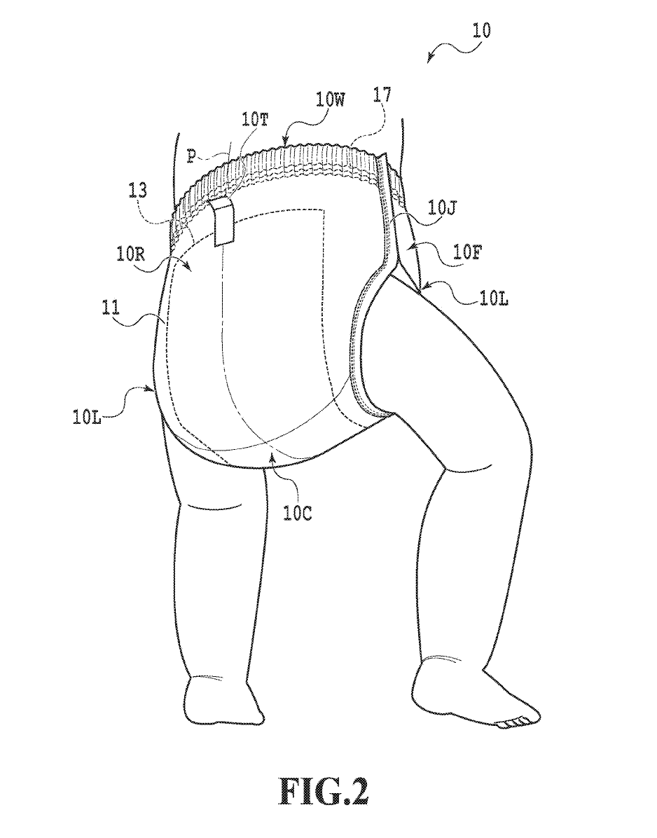

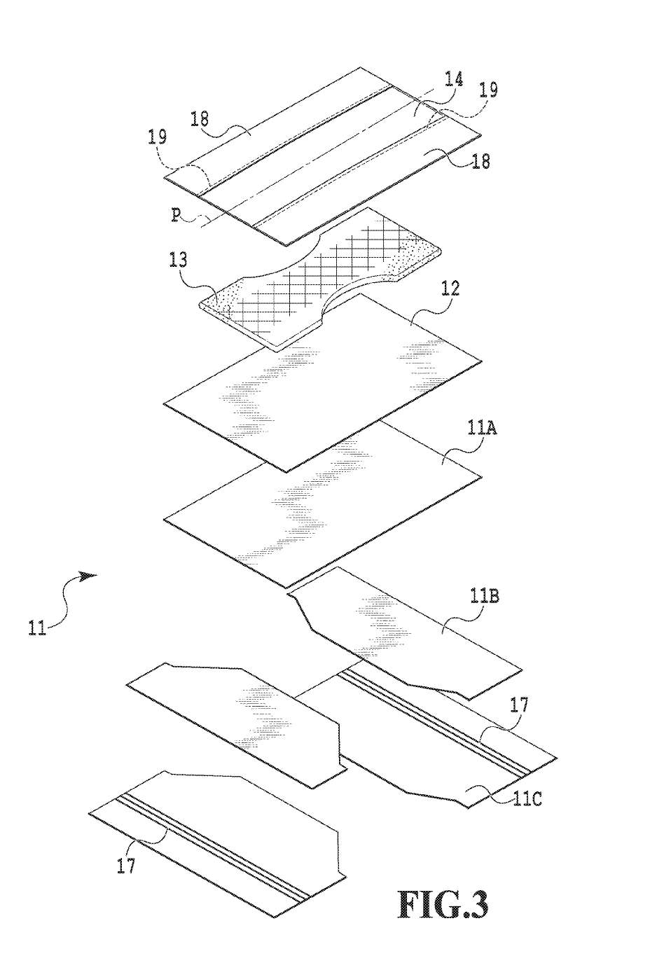

FIG. 1 is a three-dimensional projection view seen from the front side and FIG. 2 is a three-dimensional projection view seen from the back side, both showing an external appearance of Embodiment 1 in which the present invention is applied to a pants-type disposable diaper. FIG. 2 shows a lower part of a baby's body seen from the back side when he/she is wearing the pants-type disposable diaper. Furthermore, FIG. 3 shows the pants-type disposable diaper shown in FIGS. 1 and 2 in an unfolded and exploded state.

A pants-type disposable diaper (hereinafter may be simply referred to as the diaper) 10 according to this embodiment includes a front body region 10F, a back body region 10R, and a crotch region 10C connecting the front body region 10F and the back body region 10R. Also, a waist opening 10W is formed by the front body region 10F and the back body region 10R surrounding a waist portion of a wearer when he/she wears the diaper. Likewise, a pair of left and right leg openings 10L are formed by lower ends of the front body region 10F and back body region 10R and the crotch region 10C surrounding thigh portions of both legs of the wearer.

As shown in FIG. 2, when the wearer wears the diaper, the front body region 10F is positioned on the abdomen side of the wearer, and the back body region 10R is positioned on the dorsal side of the wearer. The crotch region 10C covers a crotch portion of the wearer, and the legs of the wearer are inserted into the pair of left and right leg openings 10L. Therefore, the leg openings 10L are positioned at positions between the bases of the legs and the thighs of the wearer.

Also, the back body region 10R of the diaper is provided with a disposal tape 10T for rolling up and holding the diaper for disposal.

An imaginary line P extends through the crotch portion from the abdomen side to the dorsal side in the central part of the diaper. To be more specific, assuming that the waist side of the diaper is the top and the crotch side thereof is the bottom, for example, the imaginary line P extends in the top-bottom direction along the surface of the diaper and also extends in the top-bottom direction on the dorsal side through the crotch portion.

In other words, the top-bottom direction is a direction along the central axis of the body from the head to the crotch of the wearer, and the imaginary line P extends along the central axis of the body.

As shown in FIG. 3, the diaper 10 according to this embodiment is formed by stacking and joining a cover sheet 11, a back sheet 12, an absorber 13, and a top sheet 14 that touches the wearer's skin in this order from outside. Furthermore, the cover sheet 11 is further divided into a pad cover sheet 11A, an inner cover sheet 11B, and an outer cover sheet 11C, which are stacked below the back sheet 12 and are all formed of thin non-woven fabric to obtain a good texture. The inner cover sheet 11B and the outer cover sheet 11C, which define the front body region 10F and the back body region 10R of the diaper 10, have their both left and right edge portions joined together to form mating portions 10J. Thus, the waist opening 10W described above is defined. The inner cover sheet 11B and the outer cover sheet 11C are divided in half at the crotch region 10C portion, and thus the pad cover sheet 11A is exposed in the crotch portion. Therefore, the pair of left and right leg openings 10L surrounding the thigh portions of both legs are defined by edge portions of the pad cover sheet 11A and edge portions of the mating portions 10J. The back sheet 12 which is liquid impermeable is attached to the pad cover sheet 11A, and the absorber 13 described above is disposed between the back sheet 12 and the top sheet 14 which is liquid permeable. The top sheet 14 is attached to the back sheet 12 with the absorber 13 interposed therebetween. Note that the inner cover sheet 11B and the outer cover sheet 11C each may be one continuous sheet that is not divided in the crotch portion. Meanwhile, the leg openings 10L may be formed into a gathered shape by rubber threads.

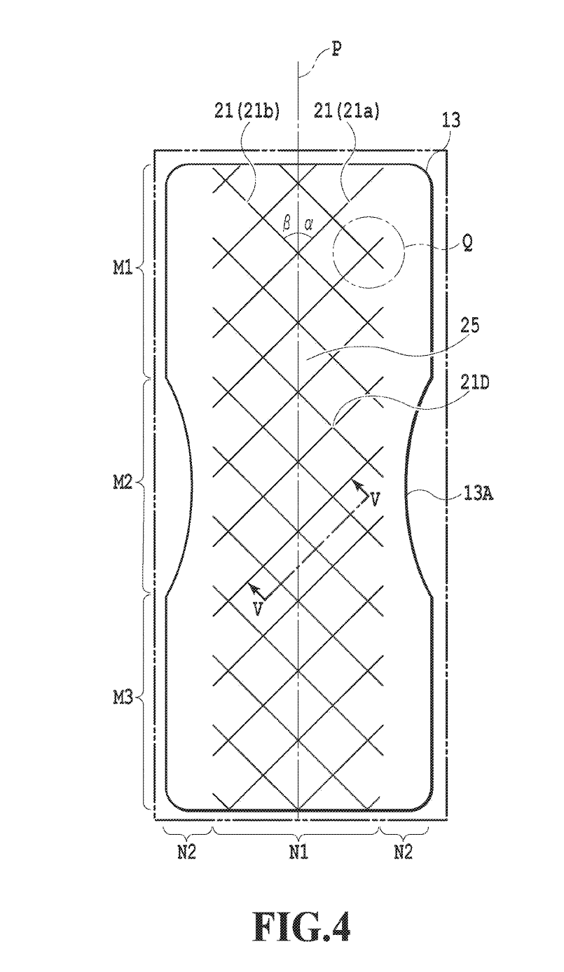

FIG. 4 is a partial top view of the crotch portion of the diaper 10 seen from the top sheet 14 side.

The absorber 13 of this embodiment positioned below the top sheet 14 is mainly made of pulp and super absorbent polymers (hereinafter also referred to as the “SAPS”). The absorber 13 has an elongated shape across the front body, the crotch, and the back body, and is divided into three portions, i.e., a front body portion M1, a crotch portion M2, and a back body portion M3. The crotch portion M2 has a pair of arc-shaped cutouts 13A formed therein corresponding to the pair of left and right leg openings 10L surrounding the thighs of the both legs. Note that the cutouts 13A do not have to be formed corresponding to the size of the absorber 13. Moreover, the absorber 13 of this embodiment has a sandglass shape with the cutouts 13A and a width narrower in the central part than at front and back ends. However, the shape of the absorber according to the present invention is not limited thereto. Assuming that the direction from the front body portion to the back body portion is the front-back (top-bottom) direction and the direction perpendicular thereto is the left-right direction, the present invention includes various shapes such as a shape having round corners at the front and back (top and bottom) ends, an oval shape extending in the front-back (top-bottom) direction, a circular shape, and a rectangular shape with approximately the same lengths in the front-back (top-bottom) and left-right directions, for example.

As shown in FIG. 4, ventilation grooves 21 are formed by regular embossing from the surface of the top sheet 14 toward the absorber 13. In this embodiment, the ventilation grooves 21 are not formed up to the ends of the absorber 13 in the width direction (left-right direction in FIG. 4) but formed in a band pattern in the center thereof. It is assumed that a region where the ventilation grooves 21 are formed is a ventilation groove formation region N1 and regions where no ventilation grooves 21 are formed on both sides thereof are ventilation groove non-formation regions N2. A breathability effect can be achieved with the ventilation grooves 21 provided at least in the center, compared with the case where no grooves are provided. It is needless to say that the ventilation grooves 21 may be provided up to the ends of the absorber 13. Therefore, the present invention may also adopt a configuration without the ventilation groove non-formation regions N2.

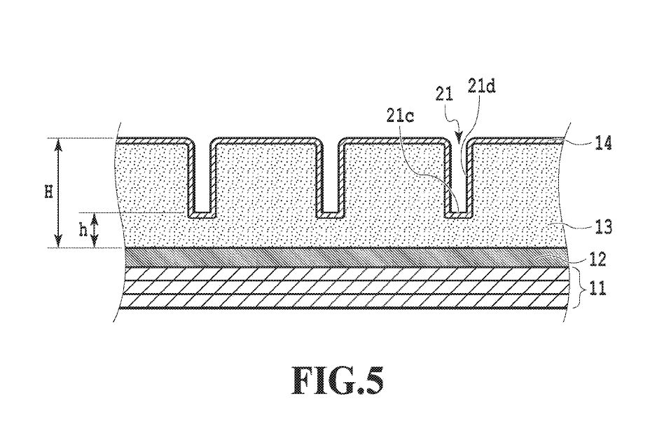

FIG. 5 is a cross-sectional view showing the crotch portion of the diaper taken along the line V-V in FIG. 4. FIG. 5 shows the cross-section of the ventilation grooves 21 extending in an oblique lattice pattern. The ventilation grooves 21 are formed by compressing the top sheet 14 and the absorber 13 at the same time from the surface (skin contact surface) of the top sheet 14, which touches the skin. To be more specific, an adhesive is applied onto the surface of the top sheet 14 that comes into contact with the absorber 13, and then the top sheet 14 and the absorber 13 are both compressed by embossing. Thus, the top sheet 14 and the absorber 13 engage with each other and are integrally joined together in the entire groove from a bottom surface (bottom) 21c to a wall surface (sidewall) 21d of the ventilation groove 21. A thickness h of the top sheet 14 and the absorber 13 in the bottom portion of the ventilation groove 21 is ⅙ of a thickness H when the compression is not performed. Therefore, the depth corresponds to about 80% of the original lamination thickness of the top sheet 14 and the absorber 13, which is deeper than when only the top sheet 14 is compressed to have a bump-recess pattern. A compression rate for the compression of the absorber is preferably about 4 to 7 times.

By forming the deep ventilation grooves 21 and integrally joining the top sheet 14 and the absorber 13 to form the sidewalls 21d as described above, the ventilation grooves 21 are made deeper and stronger than the bump-recess pattern formed only in the top sheet. Therefore, the sidewall 21d portion of the ventilation groove 21 is not easily collapsed under the load of sitting and fill up the ventilation groove 21.

Meanwhile, the formation of the ventilation grooves 21 causes voids to be formed intermittently across the absorber 13 in the surface of the diaper that touches the skin. Thus, such voids can ensure sufficient breathability.

Note that the sidewall 21d of the ventilation groove 21 may extend perpendicularly to the bottom 21c as shown in FIG. 5, or may be obliquely tilted. More specifically, the ventilation groove 21 may have a V-shaped cross section that is narrowed toward the bottom, or the like.

Here, the ventilation grooves 21 may have a continuous groove structure formed by compression at a constant pressure or may be formed in an emboss pattern such as dots.

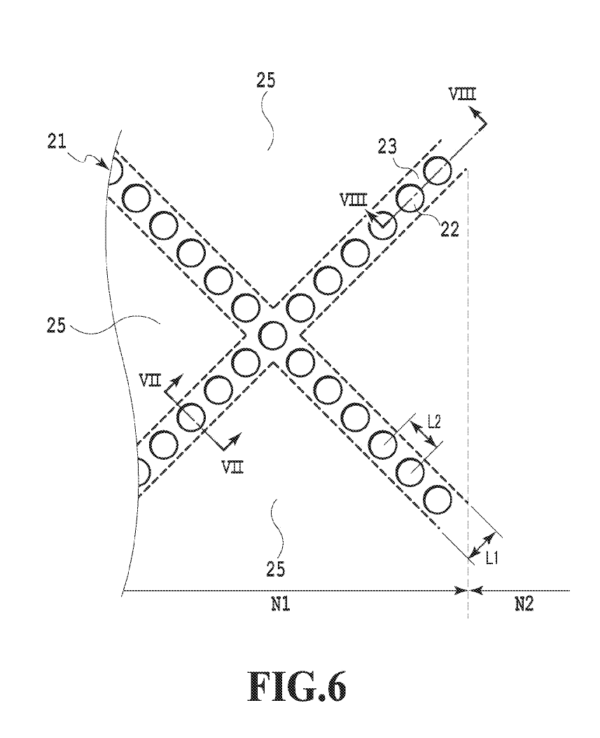

FIG. 6 shows an example where the ventilation grooves 21 are formed in an emboss pattern of dots in a range Q indicated by the dashed-dotted line across the ventilation groove formation region N1 and the ventilation groove non-formation region N2 in FIG. 4. In the emboss pattern of dots, recess portions 22 are arranged by intermittent compression from the surface of the top sheet 14 to the absorber 13. By arranging the recess portions 22 at certain intervals, the absorber 13 near the recess portions 22 is depressed, resulting in a dent portion 23 recessed from a section (hereinafter also referred to as the “main absorptive region”) 24 with no emboss pattern. Therefore, the ventilation groove 21 including the recess portions 22 and the dent portion 23 is formed by continuously forming the recess portions 22.

A width L1 of the ventilation groove 21 is about 6 mm, and a distance L2 between the centers of the adjacent recess portions is about 6 mm. It is preferable that 1 mm≦L1≦8 mm and 1 mm≦L2≦8 mm.

When the absorber 13 that is not compressed in the center of the main absorptive region 24 has a thickness of 3 to 3.6 mm and a density of 0.16 g/cm³, the absorber 13 has a thickness of 0.5 to 0.6 mm and a density of 0.96 g/cm³ in the recess portion 22 and has a thickness of 1 to 1.2 mm and a density of 0.48 g/cm³ in the dent portion 23. When a difference in density of the absorber between the recess portion 22 and the center of the main absorptive region 24 is about 6 times as described above, the arrangement interval L2 between the recess portions is preferably adjusted so that the density difference between the recess portion 22 and the dent portion 23 is about 2 to 3 times.

In such a case of the ventilation grooves 21 formed by intermittent embossing, again, the compression rate is preferably about 4 to 7 times in the recess portion 22. Also in the gap 23 portion between the recess portions 22, the recess portions 22 are preferably compressed at a compression rate that achieves sufficient compression of the absorber 13. Note that, as in the case of Embodiment 1, the compression rate is not limited to the above range but includes other ranges depending on the material of the absorber 13 and a liquid to be absorbed.

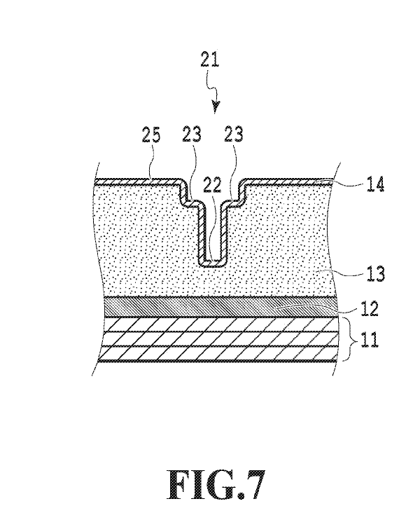

FIG. 7 is a cross-sectional view taken along the line VII-VII in FIG. 6.

As shown in FIG. 7, the embossing is compressing the top sheet 14 and the absorber 13 together from the surface of the top sheet 14 by interposing an adhesive between the top sheet 14 and the absorber 13. The top sheet 14 and the absorber 13 completely stick together in the recess portions 22. To be more specific, such compression allows some fibers of the top sheet 14 to engage with surface fibers of the absorber 13. Therefore, as in the case where the ventilation grooves 21 are continuously formed by compression, the recess portions 22 and the dent portion 23 are sufficiently deep and thus are not easily collapsed even under the load of sitting and fill up the ventilation groove 21.

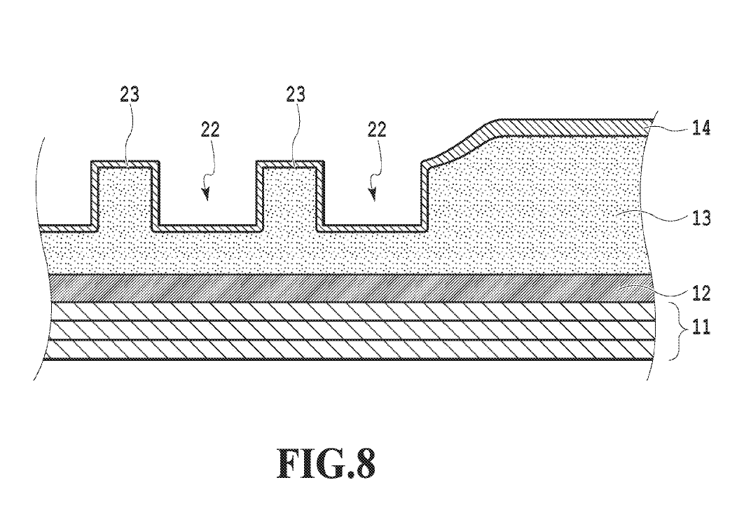

FIG. 8 is a cross-sectional view taken along the line VIII-VIII in FIG. 6.

As shown in FIG. 8, the dent portions 23 are positioned higher than the recess portions 22 but are positioned lower than the portion where no ventilation grooves 21 are formed. Therefore, by forming the recess portions 22 at predetermined intervals, obliquely extending grooves can be formed as in the case of continuous embossing.

Meanwhile, referring back to FIG. 4, the ventilation grooves 21 in this embodiment include: first ventilation grooves 21a extending while being tilted to one side (to the right in FIG. 4) in the width direction of the absorber; and second ventilation grooves 21b extending while being tilted to the other side (to the left in FIG. 4). As for the position of the imaginary line P on the absorber 13, the imaginary line P extends from the upper end of the front body portion M1 to the lower end of the back body portion M3. To be more specific, as shown in FIG. 4, when the absorber 13 has the elongated shape, the imaginary line P extends in the longitudinal direction. The ventilation grooves 21 extend while being tilted with respect to the imaginary line P. More specifically, with respect to the direction along the side about the imaginary line P, for example, the longitudinal direction, the first ventilation grooves 21a are tilted at an angle α to one side and the second ventilation grooves 21b are tilted at an angle β to the other side. The angle α and the angle β may be the same or different from each other. Moreover, in FIG. 4, the first ventilation grooves 21a are tilted at the same angle and disposed at regular intervals. In other words, the first ventilation grooves 21a are disposed parallel to each other at regular intervals. However, the present invention is not limited thereto, and also includes those with different intervals between grooves and those with different tilt angles of the grooves. The same goes for the second ventilation grooves 21b.

Note that the absorber 13 is not limited to the elongated shape but may have a shape with equal length and breadth such as a square. In this case, again, the first and second ventilation grooves 21a and 21b extend while being tilted with respect to the direction along the side.

By providing the ventilation grooves 21 while being tilted with respect to the imaginary line P, the breathability can be ensured by various body movements.

More specifically, the diaper is greatly affected by pulling force caused by back and forth movement of the legs, such as walking and crawling of a baby, among the force applied in various directions to the diaper according to the body movement. The crotch portion is obliquely pulled by alternate forward movement of the left and right legs. Therefore, the absorber is likely to be obliquely twisted and deformed. Particularly, the diaper is more likely to stick to the skin and get damp when bulging by absorbing urine. Furthermore, when the diaper is twisted and shifted to the center portion of the crotch by the back and forth movement of the legs, the breathability in the crotch portion is reduced.

However, the diaper in this embodiment suppresses the deformation of the absorber 13 with the back and forth movement of the legs by compressing the top sheet 14 and the absorber 13 together to form the obliquely extending ventilation grooves 21. Furthermore, even when the absorber 13 bulges by absorbing urine, a space is always maintained between the diaper and the skin to ensure the breathability by the ventilation grooves disposed at regular intervals in the oblique direction. Meanwhile, since the main absorptive region 24 surrounded by the ventilation grooves 21 bulges, the sections of the main absorptive region 24 bulge in an aligned state in the oblique direction even when the absorber 13 bulges by absorbing urine. Even if the diaper is obliquely pulled by the leg movement, the diaper comes into contact with the skin with a space therebetween since the main absorptive region is obliquely divided by the ventilation grooves 21.

Accordingly, ventilation paths are always secured in the oblique direction. Therefore, the crotch portion can be always kept in a breathable state.

For example, if the ventilation grooves are only in the direction along the imaginary line P, the portion surrounded by the ventilation grooves is formed into a stripe pattern. The absorber in the stripe pattern portion is broken or twisted by the oblique force caused by the back and forth movement of the legs. Then, even when the diaper absorbs urine, the urine stays in the broken portion of the absorber, causing the absorber to greatly bulge and stick to the skin. Therefore, even when the ventilation grooves are provided, the greatly bulging absorber blocks the ventilation grooves and prevents air from flowing through the entire crotch. As a result, the breathability is reduced compared with the oblique grooves. As described above, the oblique ventilation grooves 21 can ensure the breathability also in the back and forth movement of the legs, and are effective in maintaining the crotch portion in a comfortable state by preventing twisting and breaking of the absorber 13.

Note that the ventilation grooves 21 do not have to extend to the ends of the diaper.

### MODIFIED EXAMPLES

Next, modified examples of this embodiment are described.

### Modified Example 1

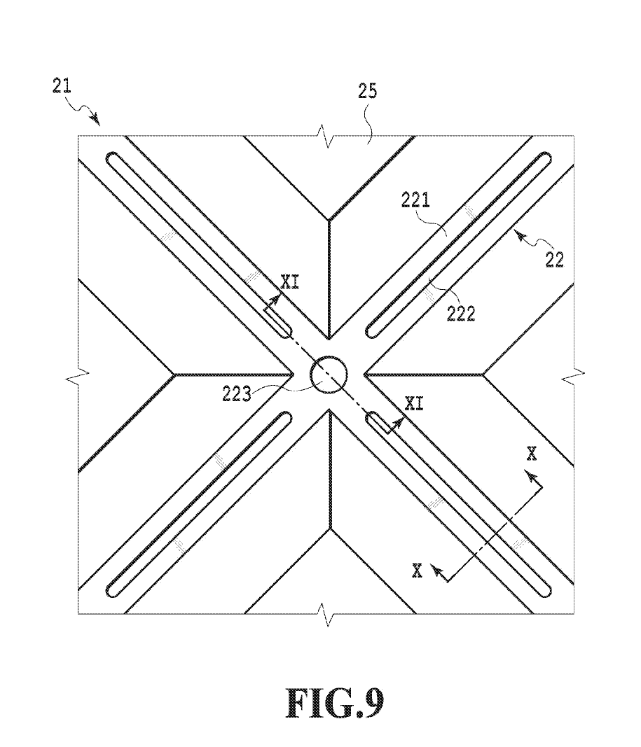

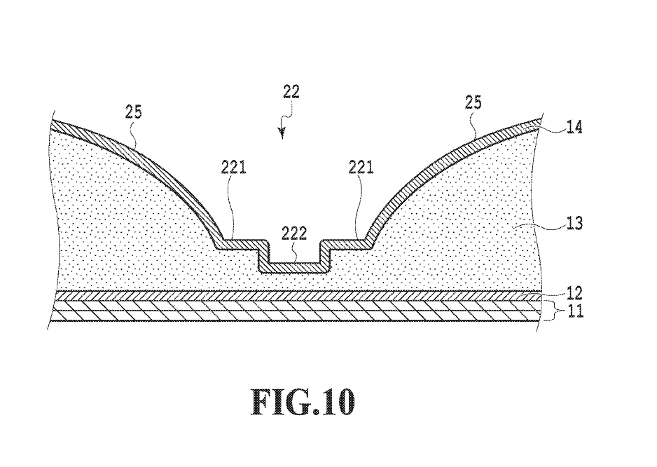

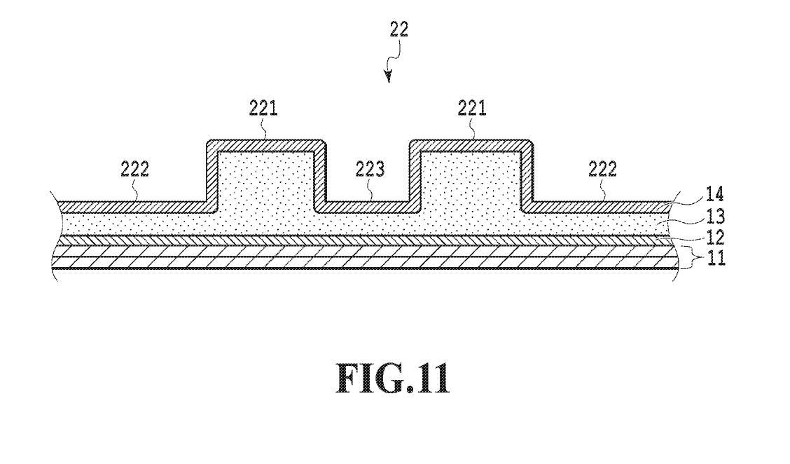

FIG. 9 is a schematic enlarged view of a part of the ventilation groove 21 in the same range Q as in FIG. 6, showing an example where the ventilation groove 21 is formed in an emboss pattern of recess portions 22 formed into a structure with two depths. FIG. 10 is a cross-sectional view of the ventilation groove 21 taken along the line X-X in FIG. 9. FIG. 11 is a cross-sectional view of the ventilation groove 21 taken along the line XI-XI in FIG. 9.

As shown in FIGS. 9 to 11, the recess portion 22 includes a first recess portion 221 continuously disposed in the ventilation groove 21 and a second recess portion 222 that is more deeply depressed than the first recess portion 221 in the center of the extending direction of the ventilation groove 21. In Modified Example 1, the planar shape of the second recess portion is oval (second recess portion 222) in a side portion of the lattice shape formed by the ventilation grooves 21, and is circular (second recess portion 223) in an intersection portion of the lattice shape.

In the recess portion 22, as shown in FIGS. 10 and 11, the second recess portion 222 is located at the deepest point and the first recess portion 221 is located at a position slightly higher than the bottom of the second recess portion 222. Thus, the recess portions 22 in Modified Example 1 are formed into a step shape with two depths by deeply compressing the absorber 13 from the surface of the top sheet 14 toward the back sheet 12.

As for embossing of such recess portions 22 formed with two depths, the first recess portions 221 are first formed by lightly pressing an embossing roll, and then the second recess portions 222 are formed after the top sheet 14 is stretched by heat transferred from the embossing roll. Thus, the adhesive force increased by the second recess portions 222 can prevent the top sheet 14 from coming off the absorber 13 while maintaining the softness of the top sheet 14 and the absorber 13. Furthermore, by forming the second recess portions 222, a clearly defined emboss pattern can be formed without deeply pressing the entire recess portions 22.

### Modified Examples 2 to 4

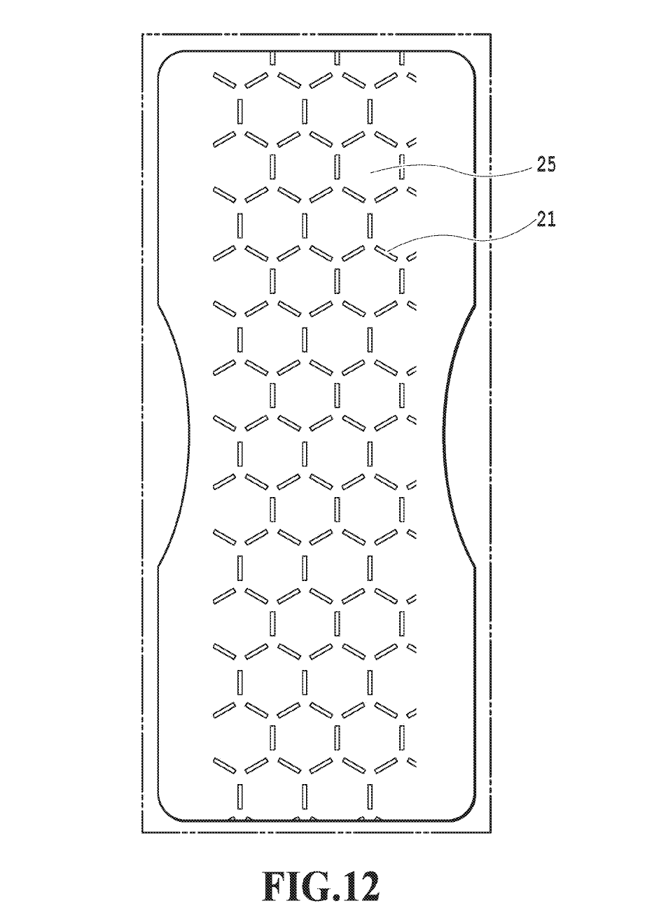

The ventilation grooves 21 may be formed not only in the oblique lattice pattern but also in a honeycomb pattern as shown in FIG. 12 (Modified Example 2). In this case, again, oblique ventilation grooves 21 are formed, although intermittently, which suppress twisting of the absorber 13 due to the back and forth movement of the legs. Thus, the breathability can be ensured without inhibiting the leg movement even when the absorber bulges by absorbing urine.

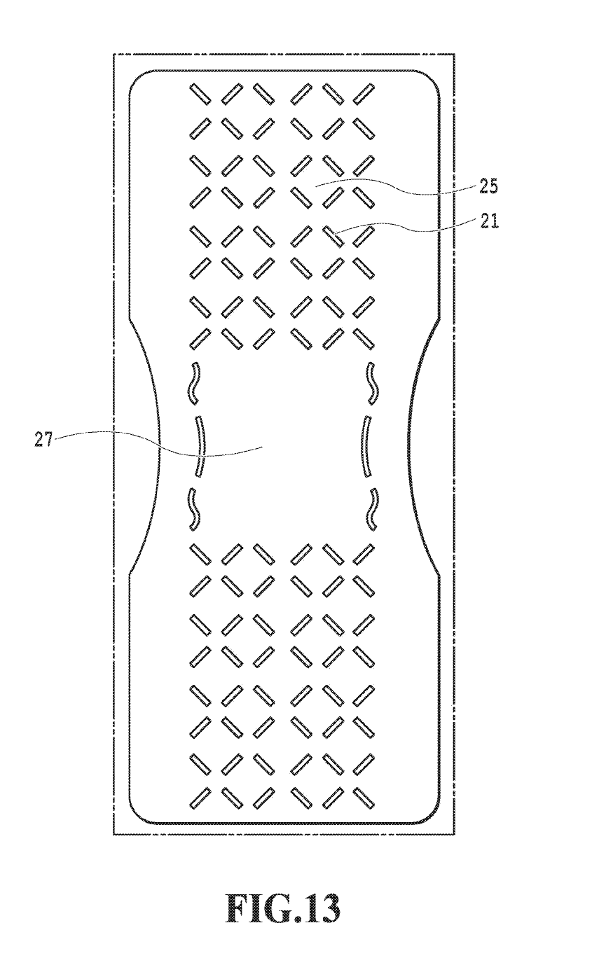

Alternatively, as shown in FIG. 13, a configuration may be adopted in which no ventilation grooves 21 are provided, in favor of the texture, at a urine discharge position 27 (Modified Example 3). Even though the breathability is reduced compared with the one provided with the ventilation grooves 21 at the urine discharge position (for example, the one shown in FIG. 4), the ventilation grooves 21 provided in front of and behind the urine discharge position increase the breathability after urination compared with the one without any ventilation grooves 21.

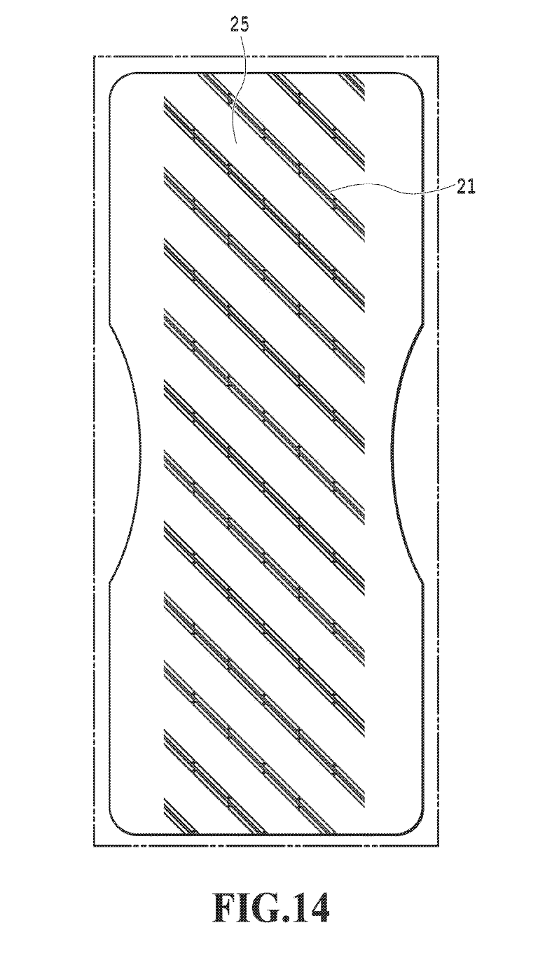

Alternatively, as shown in FIG. 14, oblique ventilation grooves 21 may be provided only in one direction (Modified Example 4). Since the main absorptive region 24 is formed in an oblique stripe pattern, the shape retention of the main absorptive region 24 is reduced compared with the oblique lattice pattern shown in FIG. 4. However, such formation of oblique ventilation grooves 21 allows the absorber 13 to follow body movements more easily and also makes the absorber resistant to twisting and breaking compared with the ventilation grooves 21 formed along the imaginary line P.

Furthermore, as for the recess portions 22, not only the circular dots but also various shapes can be used, such as a semicircular shape, a rectangular shape, and a triangular shape.

In addition, the diaper is not only the one in which the top sheet 14 is disposed directly on the absorber 13, but may also be one in which the top sheet 14 is disposed on the absorber 13 after the absorber 13 is wrapped in a hydrophilic sheet. Alternatively, a liquid diffusion sheet to improve the liquid diffusivity may be provided as needed between the top sheet 14 and the hydrophilic sheet or between the hydrophilic sheet and the absorber 13. This liquid diffusion sheet facilitates diffusion of body fluids. Meanwhile, the hydrophilic sheet may be attached so as to cover the absorber 13 or may be simply disposed on the front and the back without covering the ends of the absorber 13.

Note that FIG. 2 shows a standing state of the lower part of the baby's body wearing the diaper 10. However, the present invention is not aimed at only the case of standing and walking, but achieves the advantageous effects for general back and forth movements of the legs, such as rolling over and crawling of a baby.

(Evaluation of Breathability)

As for the breathability of the diaper, the diaper of this embodiment including the ventilation grooves 21 is compared with a conventional diaper without grooves.

(Evaluation Method)

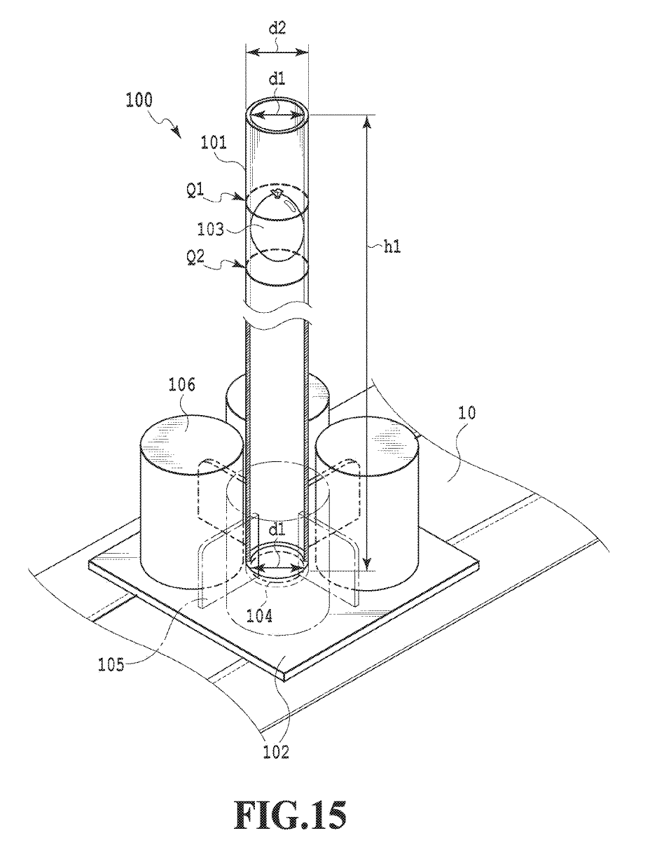

As shown in FIG. 15, a measurement device 100 includes a cylinder 101, a bottom plate 102, a water balloon 103 with 17 ml of water injected therein, and supports 105. The cylinder 101 is a transparent tube with an inside diameter d1 of 26 mm, an outside diameter d2 of 30 mm, and a height of 700 mm, which stands perpendicularly to the bottom plate 102. At the junction with the cylinder 101, the bottom plate 102 has an opening 104 with the same diameter d1 along the inside diameter of the cylinder 101. The bottom plate 102 is 100 mm square. The cylinder 101 is fixed by four supports 105 so as to stand upright on the bottom plate 102. During measurement, four weights 106, each weighing 190 g, are used to press the bottom plate 102 from above.

The measurement is performed as follows.

(1) The inside of the cylinder 101 is soaked with enough cooking oil.

(2) Next, the measurement device 100 is placed on a board such as a desk and a drawing board, with the bottom plate 102 as the bottom. In this placement state, the water balloon 103 is put into the cylinder 101 from the upper end (end positioned at the top in the vertical direction during measurement) of the cylinder 101. In this event, time required for the water balloon 103 to move from a measurement start point Q1 located at a position 250 mm away from the upper end of the cylinder 101 to a measurement end point Q2 located at a position 550 mm away from the upper end thereof is measured (measurement in a blank state). The measurement is performed using a timer or the like that starts and stops when optical sensors provided at the measurement start point Q1 and the measurement end point Q2, respectively, detect the water balloon 103. Alternatively, the measurement is performed by operating a stopwatch or the like with visual observation.

(3) Then, the measurement device 100 is placed on the diaper 10 to be measured. Thereafter, the weights 106 are evenly placed and fixed on the measurement board 102. The weights are assumed to be the load applied when the diaper is worn. By applying the load with the weights from the top sheet side of the diaper, it is assumed that the absorber is somewhat collapsed in the height direction.

(4) The water balloon 103 with 17 ml of water injected therein is soaked in the cooking oil and then excess oil is drained for 2 seconds. Thereafter, the water balloon 103 is put in from the upper end of the cylinder 101. Then, as in the case of the measurement in the blank state, time required for the water balloon 103 to move from the measurement start point Q1 to the measurement end point Q2 is measured.

(5) A value obtained by subtracting the measured value in the blank state (2) from the measured value obtained in (4) is set as the breathability of the diaper 10 to be measured.

The air in the cylinder 101 into which the water balloon 103 is put passes below the bottom plate 102 from the opening 104 at the lower end and then flows out. However, when the measurement device is placed on the diaper, the diaper resists the airflow and stagnates the airflow inside the cylinder 101 compared with the blank state. In addition, with the worse breathability of the diaper, the air inside the cylinder 101 resists the water balloon 103, and therefore, it takes time for the water balloon 103 to fall. This leads to a conclusion that the shorter the time required for the water balloon 103 to fall, the better the breathability of the diaper.

Using the measurement method as described above, the conventional diaper without ventilation grooves is compared with the diaper 10 in which the ventilation grooves are formed in the oblique lattice pattern shown in FIG. 4 in the longitudinal direction of the absorber 13, for the breathability.

Table 1 shows the result of the measurement performed by placing the measurement device 100 in the center of each of the regions obtained by dividing each of the diapers into the abdomen side, the crotch, and the dorsal side, i.e., each of the regions of the front body portion M1, the crotch portion M2, and the back body portion M3 in FIG. 4.

The values are in seconds.

TABLE 1

| | Front Body | | Back Body |
|---|---|---|---|
| | Portion | | Portion |
| | (Abdomen | Crotch | (Dorsal |
| | Side) | Portion | Side) |
| Embodiment 1 (with | 0.6 | 0.7 | 0.6 |
| ventilation grooves in | | | |
| oblique lattice | | | |
| pattern) | | | |
| Comparative Example | 5.0 | 5.6 | 5.6 |
| (without ventilation | | | |
| grooves) | | | |

As shown in Table 1, the diaper of Embodiment 1 with the ventilation grooves has the breathability improved compared with the conventional diaper without ventilation grooves, in all the regions of the front body portion, the crotch portion, and the dorsal side portion.

By providing the ventilation grooves in the diaper by compressing the top sheet and the absorber together as described above, the breathability is improved in all the regions of the diaper, thereby suppressing unpleasant dampness inside the diaper and occurrence of irritation after urination.

Meanwhile, the ventilation grooves secure the ventilation paths without being collapsed even under the load applied by the four weights. In other words, the ventilation grooves of this embodiment are not collapsed even under the load applied by the wearer sitting.

Note that the structure of the diaper 10 to be the target of the present invention is not limited to the pants-type as described above. The present invention is applicable to any configuration as long as the diaper includes the configuration of the absorbent article according to claims. For example, the present invention is also applicable to well-known unfolding-type disposable diaper, urine leakage prevention pad, and the like.

In addition, the present invention is not limited to a diaper for baby, but is applicable to various absorbent articles such as a diaper for adult and the urine leakage prevention pad.

### Embodiment 2

Next, description is given of a structure of an absorber portion according to Embodiment 2 of the present invention.

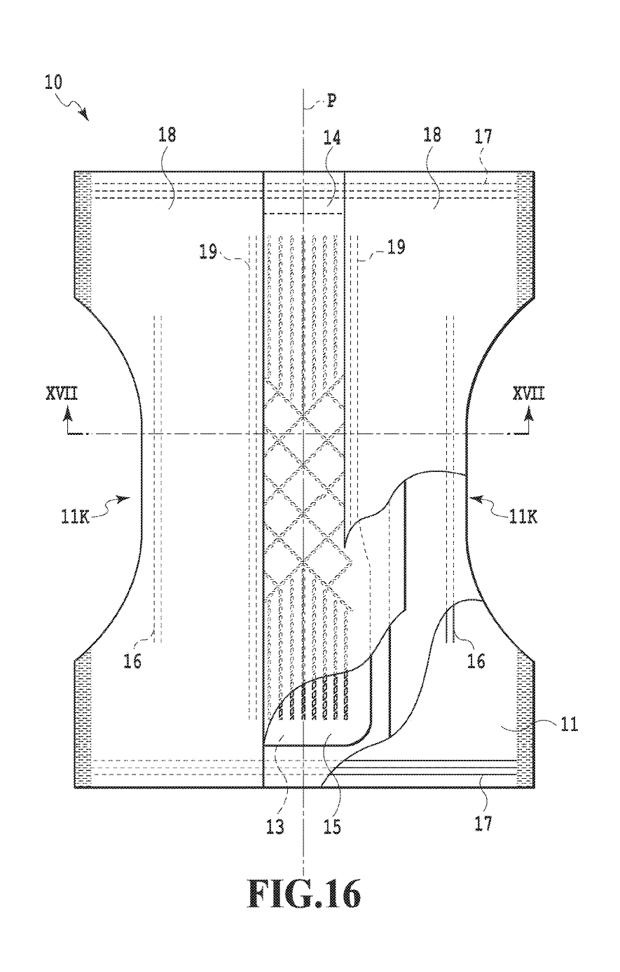

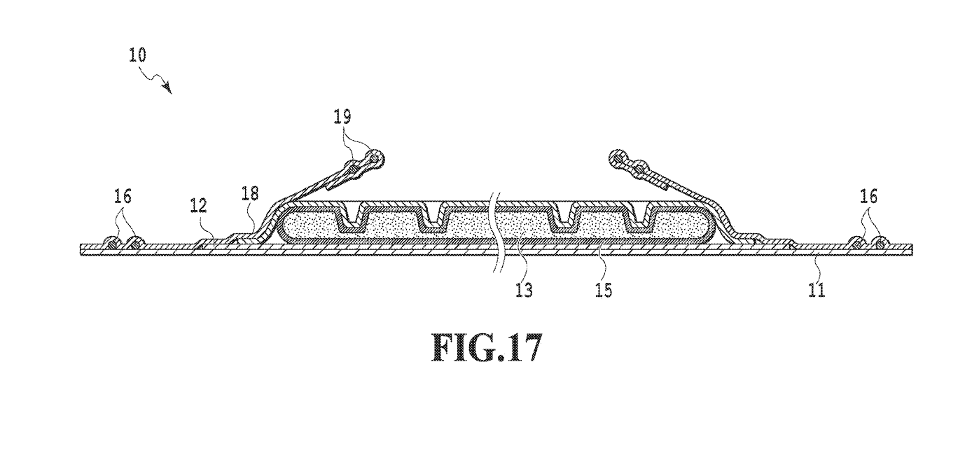

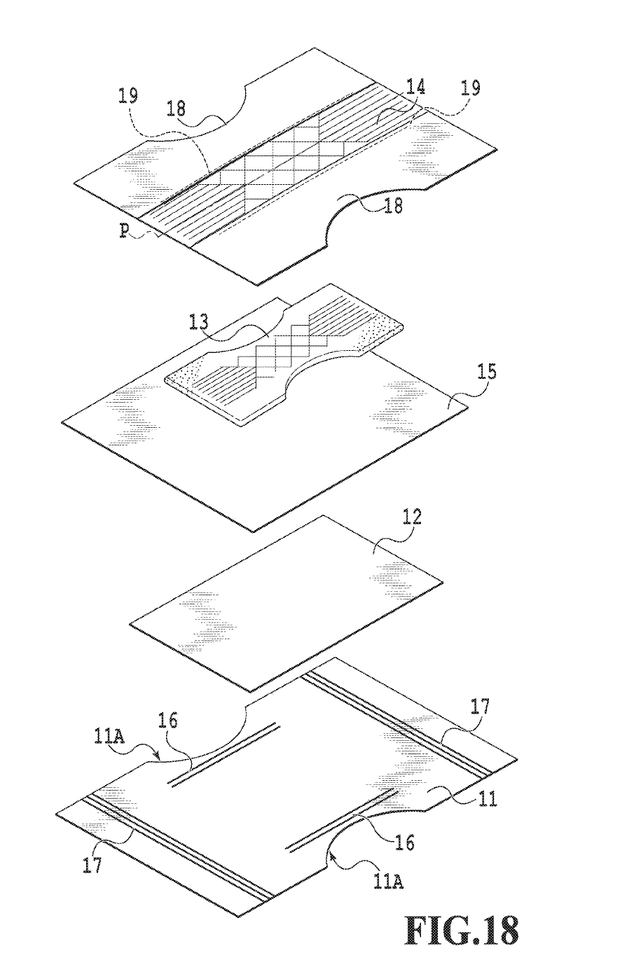

FIG. 16 is a plan view showing a diaper 10 according to Embodiment 2 seen from the skin contact surface side in an unfolded state of the diaper shown in FIG. 1. FIG. 17 is a cross-sectional view taken along the line XVII-XVII in FIG. 16. FIG. 18 shows the pants-type disposable diaper shown in FIGS. 1 and 16 in an unfolded and exploded state.

As shown in FIGS. 16 to 18, the diaper 10 according to this embodiment is formed by stacking and joining a cover sheet 11, a liquid impermeable back sheet 12, an absorber 13 wrapped in a core wrap (tissue) 15 that is a hydrophilic thin sheet, and a liquid permeable top sheet 14 that touches the wearer's skin in this order from outside when seen from the wearer's skin side.

Note that an absorbent article attached to an underwear or the like to absorb liquids can also be formed of the back sheet 12, the absorber 13 wrapped in the core wrap 15, and the top sheet 14.

The cover sheet 11 is formed of thin non-woven fabric to obtain a good texture and is attached to the back sheet 12. Also, in the center of both left and right edge portions of the cover sheet 11 in the width direction, a pair of arc-shaped cutouts 11K to be the leg openings 10L in FIG. 1, respectively, are formed. The longitudinal direction of the back sheet 12 is approximately parallel to the imaginary line P, and the back sheet 12 is attached onto the cover sheet 11 so that the imaginary line P passes through the center thereof in the width direction or the vicinity thereof.

The absorber 13 of this embodiment has the elongated shape and is wrapped in the core wrap 15 (tissue) that is the hydrophilic thin sheet having ends glued together, for example, to maintain the shape. The absorber 13 wrapped in the core wrap 15 is disposed between the back sheet 12 and the top sheet 14. The top sheet 14 of this embodiment is attached to the back sheet 12 through the absorber 13 wrapped in the core wrap 15. Note that, although the absorber 13 wrapped in the core wrap is used in this embodiment, the absorber in the present invention does not have to be wrapped in the core wrap.

At positions adjacent to the pair of cutouts 11K in the cover sheet 11 and at two ends of the cover sheet 11 in the longitudinal direction, a rubber thread 16 to forma leg gathering and a rubber thread 17 to form a waist gathering are attached in a stretched state.

A pair of side sheets 18 to form a three-dimensional gathering is provided at both left and right edge portions of the top sheet 14 in the width direction. The pair of side sheets 18 have their outer edge portions formed into the same shape as that of the pair of cutouts 11K in the cover sheet 11. The side sheets 18 are members for preventing lateral leakage of urine discharged by the wearer by standing up along both left and right edges of the absorber 13 when the diaper is worn. In the side sheets 18, rubber threads 19 as three-dimensional gathering elastic members are disposed in a stretched state such that inner edge portions thereof folded to the absorber 13 side and held. The rubber threads 19 stand up in the skin contact direction of the wearer upon contraction thereof. The three-dimensional gathering can adopt a heretofore known configuration used in the conventional disposable diaper. For example, the rubber threads 19 can be formed by interposing and fixing the three-dimensional gathering elastic members in the stretched state between water-repellent sheet layers.

As shown in FIG. 16, the side sheets 18 are drawn in the longitudinal direction by the contraction of the rubber threads 19. Then, as shown in FIG. 17, the inner edge portions thereof stand up and form the three-dimensional gathering.

Note that, in the diaper 10 according to this embodiment, the absorber 13 is elongated along the imaginary line P, and the longitudinal direction thereof is parallel to the imaginary line P. A direction perpendicular to the imaginary line P is the width direction. Note that the ratio of the length in the longitudinal direction to the length in the width direction of the diaper is not limited to that of this embodiment. The ratio is changed as appropriate according to the body type of the wearer.

Next, description is given of the structure of the absorber portion according to this embodiment.

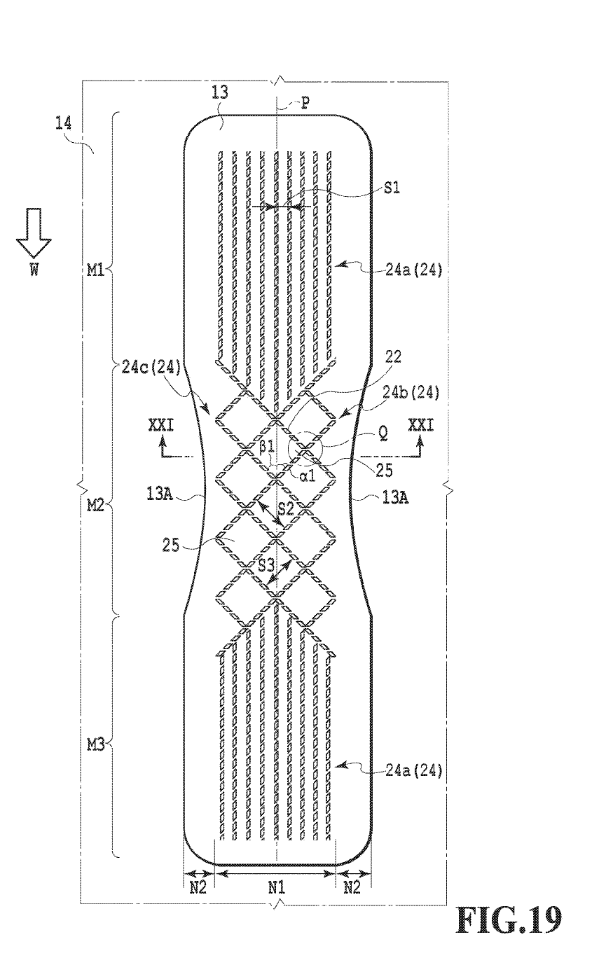

FIG. 19 is a partial top view seen from the top sheet 14 side, showing the portion where the absorber 13 and the top sheet 14 are positioned.

The absorber 13 of this embodiment positioned below the top sheet 14 is mainly made of pulp and super absorbent polymers (hereinafter also referred to as the “SAPS”). The absorber 13 has an elongated shape across the front body, the crotch, and the back body, and is divided into three portions, i.e., a front body portion M1, a crotch portion M2, and a back body portion M3. The crotch portion M2 has a pair of arc-shaped cutouts 13A formed therein corresponding to the pair of left and right leg openings 10L surrounding the thighs of the both legs. Note that the cutouts 13A do not have to be formed corresponding to the size of the absorber 13. Moreover, the absorber 13 of this embodiment has a sandglass shape with the cutouts 13A and a width narrower in the central part than at front and back ends. However, the shape of the absorber according to the present invention is not limited thereto. Assuming that the direction from the front body portion M1 to the back body portion M3 is the front-back (top-bottom) direction and the direction perpendicular thereto is the left-right direction, the present invention includes various shapes such as a shape having round corners at the front and back (top and bottom) ends, an oval shape extending in the front-back (top-bottom) direction, a circular shape, and a rectangular shape with approximately the same lengths in the front-back (top-bottom) and left-right directions, for example.

Here, the center region of the absorber 13 according to the present invention means a region that first comes into contact with a liquid such as urine, for example, and a region in the vicinity thereof. Note that the center region of the absorber 13 can be changed according to the physical build, gender, and the like of the wearer of the absorbent article.

Next, description is given of an emboss pattern formed from the top sheet 14 to the absorber 13 according to this embodiment.

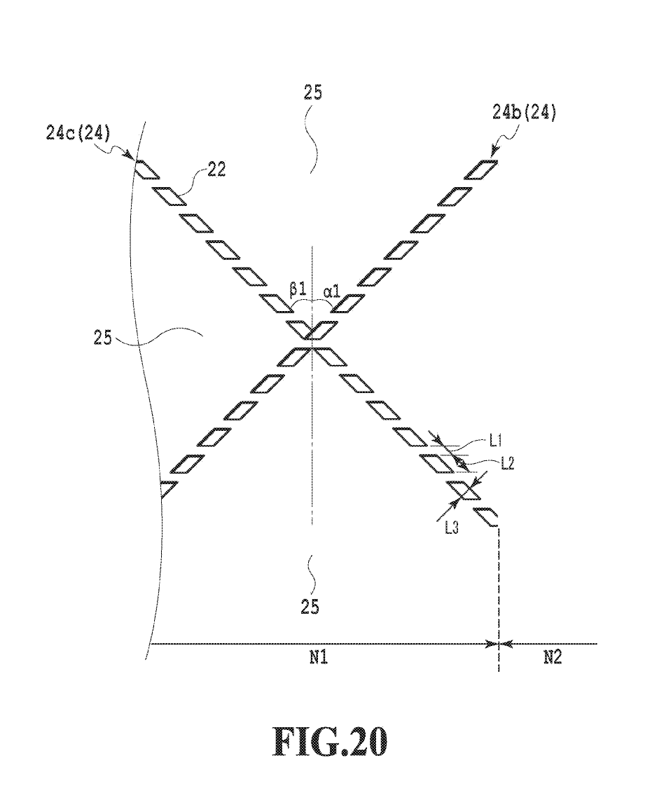

As shown in FIGS. 19 and 20, the diaper 10 has a compressed line formation region (emboss pattern formation region) N1, which is compressed by regular embossing from the surface of the top sheet 14 to the absorber 13. In the compressed line formation region N1, recess portions 22 are formed by compression. In the front body portion M1 and back body portion M3 of the compressed line formation region N1, compressed lines 24 are formed in a linear pattern (third pattern) extending in the longitudinal direction of the absorber when seen from a distance by the arrangement of the recess portions 22. Also, in the crotch portion M2 of the compressed line formation region N1, compressed lines 24 are formed in an oblique lattice pattern (first and second patterns) when seen from a distance by the arrangement of the recess portions 22.

As shown in FIG. 20, the compressed lines 24 are formed by arranging the recess portions 22 at regular intervals. The compressed lines 24 are divided into: first compressed lines 24a (third pattern) formed in the front body portion M1 and the back body portion M3; and second compressed lines 24b (first pattern) and third compressed lines 24c (second pattern) formed in the crotch portion M2. The first compressed lines 24a extend in the top-bottom direction parallel to the imaginary line P in FIG. 19, which is the direction in which the two end regions of the absorber 13 face each other across the center region thereof. The first compressed lines 24a are arranged parallel to each other. On the other hand, the second compressed lines 24b are tilted at a tilt angle α1 to the right in FIG. 19 that is a second direction with respect to the imaginary line P. Meanwhile, the third compressed lines 24c are tilted at a tilt angle β1 to the left in FIG. 19 that is a third direction with respect to the imaginary line P.

In this embodiment, an angle (α1+β1) formed by the second compressed line 24b and the third compressed line 24c is a right angle 90°, and the tilt angles α1 and β1 are the same angle. However, the present invention is not limited thereto. Although not shown in FIG. 20, the recess portions 22 are formed by compressing the core wrap and the absorber 13 together from the surface of the top sheet 14.

In this embodiment, by arranging the compressed lines 24 in the oblique lattice pattern in the crotch portion M2, the absorber 13 can be prevented from being twisted by the back and forth movement of the legs. More specifically, since the second compressed lines 24b and the third compressed lines 24c are provided, the absorber 13 is deformed along the compressed lines 24 against the force applied in the oblique directions by the back and forth movement of the legs. Thus, the twisting of the absorber 13 can be suppressed. Moreover, the compressed lines 24 in the crotch portion M2 increase the stiffness of the absorber 13. Thus, the absorber 13 can be suppressed from being broken by the pulling forces in the oblique directions by the leg movements.

Furthermore, in this embodiment, by arranging the compressed lines 24 in a linear pattern in the front body portion M1 and the back body portion M3, air (moisture) inside the diaper 10 is easily released to the outside and also the outside air is easily taken into the diaper from the waist portion positioned on the dorsal side or abdomen side of the wearer of the diaper 10. This facilitates the movement of the air between the inside and the outside of the diaper 10. Thus, the breathability of the diaper 10 can be improved. Moreover, by arranging the compressed lines 24 in a linear pattern as described above, the absorber 13 follows the shape of the waist portion of the diaper 10. Thus, the fitting of the wearer can be improved.

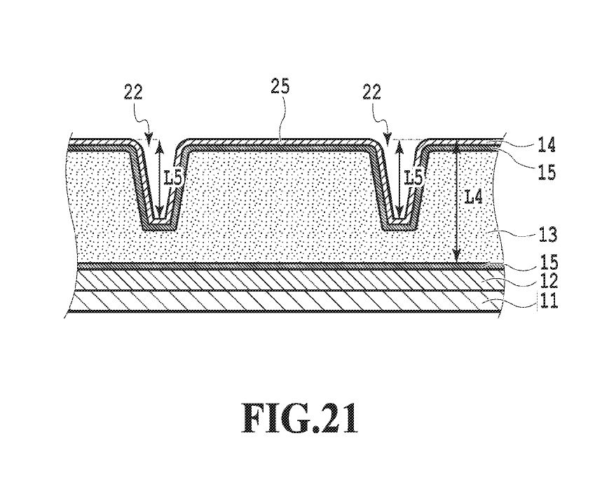

FIG. 21 is a cross-sectional view of the absorber 13 taken along the line XXI-XXI in FIG. 19. As shown in FIG. 21, the recess portions 22 are formed by compressing the top sheet 14 and the absorber 13 together with an adhesive interposed between the top sheet 14 and the absorber 13. By compressing the top sheet 14 and the absorber 13 from the surface of the top sheet 14, the recess portions 22 maintain their shape and the compressed lines 24 are not collapsed even under the body weight of the wearer such as sitting. Therefore, the diaper 10 according to this embodiment can diffuse body fluids and maintain the breathability by utilizing the compressed lines 24.

Note that, as shown in FIG. 19, the compressed lines 24 (emboss pattern) are not formed in both left and right edge portions of the absorber 13 in the width direction. Therefore, in the absorber 13, compressed line non-formation regions (emboss pattern non-formation regions) N2 are positioned on both sides of a compressed line formation region (emboss pattern formation region) N1. The absorber 13 is thus formed to prevent body fluids from leaking from the leg openings 10L through the compressed lines 24. Likewise, no compressed lines 24a are formed also at two ends of the absorber 13 in the longitudinal direction. Thus, the body fluids can be prevented from leaking from the waist opening 10W on the abdomen side or the dorsal side through the compressed lines 24a.

As shown in FIG. 19, the first compressed lines 24a are arranged parallel to each other. Likewise, the second compressed lines 24b are arranged parallel to each other, and the third compressed lines 24c are arranged parallel to each other. Meanwhile, the first compressed lines 24a (third pattern) arranged in the front body portion M1 and the back body portion M3 have their ends on the center region (crotch portion M2) side communicated with the second compressed lines 24b (first pattern) or the third compressed lines 24c (second pattern). However, the intervals between the adjacent compressed lines are not equal. An interval S2 between the second compressed lines 24b in the crotch portion M2 is two times larger than an interval S1 in the front body portion M1 and the back body portion M3. As for the third compressed lines 24c, again, an interval S3 in the crotch portion M2 is two times larger than the interval S1 in the front body portion M1 and the back body portion M3. However, the relationship of the ratio of the interval S1 to the intervals S2 and S3 is not limited to that in this embodiment.

In the recess portions 22 and the compressed lines 24 formed by the arrangement thereof, a liquid is not absorbed into the bottom, and most of the liquid flows off through the lines while some of the liquid is absorbed from the sidewalls and the like of the recess portions 22. On the other hand, the main absorptive region 25 surrounded by the compressed lines 24 can sufficiently absorb the liquid since the absorber 13 is not compressed therein.

The dimensions of the respective portions in the emboss pattern shown in FIG. 19 are as follows.

As shown in FIG. 21, a thickness L4 of the uncompressed portion such as the main absorptive region 25 in the top sheet 14 and the absorber 13 is about 4 mm. The thickness L4 is preferably within a range of 2 mm to 6 mm. A depth L5 of the deepest portion of the recess portion 22 is about 3 mm. The depth L5 is preferably about 1 mm to 5 mm.

As shown in FIG. 20, an interval L1 between the adjacent recess portions 22 among the recess portions 22 that form the compressed line 24 is about 2 mm. The interval L1 is preferably within a range of 1 mm to 5 mm. Each of the recess portions 22 has a length L2 of about 2.4 mm. The length L2 is preferably within a range of 1 mm to 50 mm. The recess portion 22 has a width L3 of about 2 mm. The width L3 is preferably within a range of 1 mm to 5 mm. Although the recess portion 22 has a parallelogram shape in FIGS. 19 and 20, the present invention is not limited thereto and various shapes can be used, such as a circular shape, an oval shape, and a triangular shape.

As shown in FIG. 19, the arrangement interval S1 between the first compressed lines 24a in the front body portion M1 and the back body portion M3 is 10 mm. The arrangement interval S1 is preferably 5 mm to 25 mm. The arrangement interval S2 between the second compressed lines 24b and the arrangement interval S3 between the third compressed lines 24c in the crotch portion M2 are 40 mm. The arrangement intervals S2 and S3 are preferably 30 mm to 50 mm. Although the arrangement intervals S2 and S3 are the same value in this embodiment, the two may be different values.

The configuration of the emboss pattern is not limited to that shown in FIG. 19. The compressed lines 24 may be tilted at different angles as long as the relationship of the arrangement of the compressed lines 24 is satisfied between the crotch portion M2 and the front and back body portions M1 and M3.

As described above, in the absorber 13 of this embodiment, the first compressed lines 24a are arranged in the longitudinal direction of the absorber 13 in the front body portion M1 and the back body portion M3, and the second and third compressed lines 24b and 24c are arranged in the oblique lattice pattern in the crotch portion M2. Thus, the wearing feeling is improved by preventing the shape deformation of the absorber 13 in the crotch portion M2. At the same time, since the tilt angles of the compressed lines 24 vary between the crotch portion M2 and the front and back body portions M1 and M3, the absorber 13 can respond to twisting in various directions. Furthermore, by arranging the compressed lines 24 in a linear pattern in the front body portion M1 and the back body portion M3, air (moisture) inside the diaper 10 is easily released to the outside and also the outside air is easily taken into the diaper from the waist portion positioned on the dorsal side or abdomen side of the wearer of the diaper 10. This facilitates the movement of the air between the inside and the outside of the diaper 10. Thus, the breathability of the diaper 10 can be improved.

### Embodiment 3

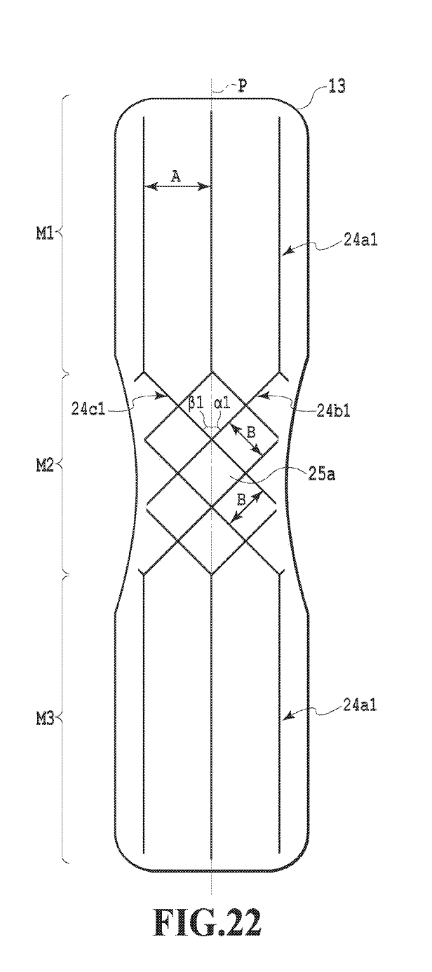

FIG. 22 is a top view seen from the top sheet 14 side, showing an absorber 13 in a disposable diaper 10 according to Embodiment 3 of the present invention. A first difference between the shape of the absorber 13 of this embodiment and the shape of the absorber 13 of Embodiment 2 is that an interval A between first compressed lines 24a1 in this embodiment is larger than the interval S1 between the first compressed lines 24a in Embodiment 2. Meanwhile, a second difference is that an interval B between second compressed lines 24b1 and between third compressed lines 24c1 in this embodiment is smaller than the interval S3 between the second compressed lines 24b and between the third compressed lines 24c in Embodiment 2.

Although, as shown in FIG. 22, tilt angles α1 and β1 of the second and third compressed lines 24b1 and 24c1 in the crotch portion M2 with respect to the imaginary line P are the same as the tilt angles α1 and β1 in Embodiment 2, the two may be different values. Moreover, although an absorptive region (uncompressed region) 25a surrounded by the second compressed lines 24b1 and the third compressed lines 24c1 in the crotch portion M2 has an area smaller than that of the absorptive region (uncompressed region) 25 in the crotch portion M2 in Embodiment 2, the area of the absorptive region 25a may be larger than or the same as that of the absorptive region 25.

As shown in FIG. 22, the arrangement interval A between the first compressed lines 24a1 in the front body portion M1 and the back body portion M3 is 40 mm. The arrangement interval A is preferably 5 mm to 50 mm. The arrangement interval B between the second compressed lines 24b1 and between the third compressed lines 24c1 in the crotch portion M2 is 25 mm. The arrangement interval B is preferably 10 mm to 40 mm. Although the arrangement intervals between the second compressed lines 24b1 and between the third compressed lines 24c1 are the same value in this embodiment, the two may be different values.

The absorber 13 of this embodiment can achieve the same effects as those achieved by the absorber 13 of Embodiment 2 as long as the second compressed lines 24b1 and the third compressed lines 24c1 form a lattice pattern and the first compressed lines 24a1 in the front body portion M1 and the back body portion M3 form a linear pattern.

Furthermore, as in the case of Embodiment 2, the absorber 13 of this embodiment has no compressed lines 24 formed in a peripheral portion thereof with the configuration described above. Thus, the absorber 13 can prevent body fluids from leaking from the leg openings 10L and the waist opening 10W.

### Embodiment 4

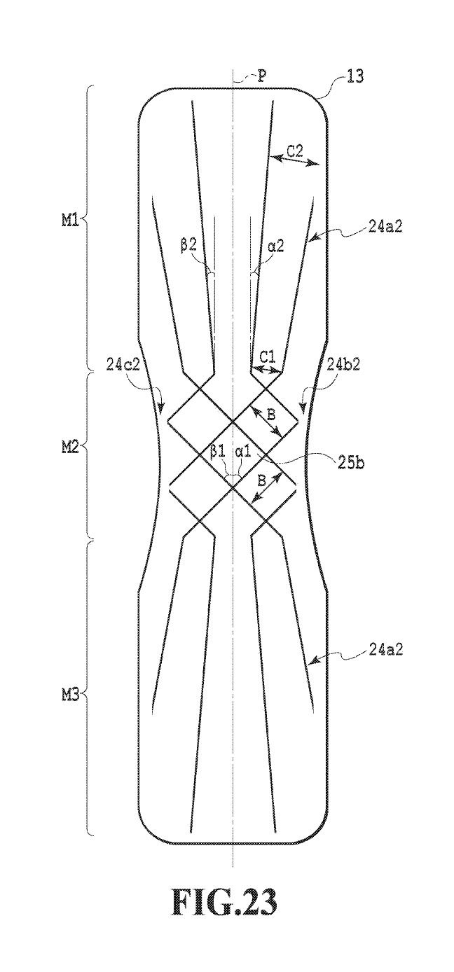

FIG. 23 is a top view seen from the top sheet 14 side, showing an absorber 13 in a disposable diaper 10 according to Embodiment 4 of the present invention. A difference in shape between the absorber 13 of this embodiment and the absorber 13 of Embodiments 2 and 3 is that first compressed lines 24a are tilted at tilt angles α2 and β2 and an interval C between first compressed lines 24a2 is increased toward two ends of the absorber 13 in the longitudinal direction.

Note that, as in the case of Embodiment 3, an interval B between second compressed lines 24b2 and between third compressed lines 24c2 is smaller than the interval S2 between the second compressed lines 24b and the interval S3 between the third compressed lines 24c.

Although, as shown in FIG. 23, tilt angles α1 and β1 of the second and third compressed lines 24b2 and 24c2 in the crotch portion M2 with respect to the imaginary line P are the same as the tilt angles α1 and β1 in Embodiments 2 and 3, the two may be different values. Moreover, although an absorptive region (uncompressed region) 25b surrounded by the compressed lines 24b2 and 24c2 in the crotch portion M2 has the same area as that of the absorptive region (uncompressed region) 25a in the crotch portion M2 in Embodiment 3, the area of the absorptive region 25b may be different from that of the absorptive region 25a. Furthermore, as shown in FIG. 23, the tilt angles α2 and β2 of the first compressed lines 24a2 with respect to the imaginary line P are 8°. The tilt angles α2 and β2 are preferably 2° to 15°.

In the front body portion M1 and the back body portion M3, an arrangement interval C1 between the first compressed lines 24a2 around the crotch portion M2 is 17 mm and an arrangement interval C2 around the two ends of the absorber 13 in the longitudinal direction is 28 mm. The arrangement interval C (C1 and C2) is preferably 10 mm to 40 mm. Note that the arrangement interval C1 is smaller than the arrangement interval C2 in this embodiment, but may be equal to or larger than the arrangement interval C2.

The arrangement interval B between the second compressed lines 24b2 and between the third compressed lines 24c2 in the crotch portion M2 is 25 mm as in the case of Embodiment 3. The arrangement interval B is preferably 10 mm to 40 mm. Although the tilt angles α2 and β2 are the same value in this embodiment, the two may be different values. Also, although the arrangement intervals B between the second compressed lines 24b2 and between the third compressed lines 24c2 are the same value in this embodiment, the two may be different values.

The absorber 13 of this embodiment can achieve the same effects as those achieved by the absorber 13 of Embodiment 2 as long as the second compressed lines 24b2 and the third compressed lines 24c2 form a lattice pattern and the first compressed lines 24a2 in the front body portion M1 and the back body portion M3 form a linear pattern.

Furthermore, as in the case of Embodiments 2 and 3, the absorber 13 of this embodiment has no compressed lines 24 formed in a peripheral portion thereof with the configuration described above. Thus, the absorber 13 can prevent body fluids from leaking from the leg openings 10L and the waist opening 10W.

### Embodiment 5

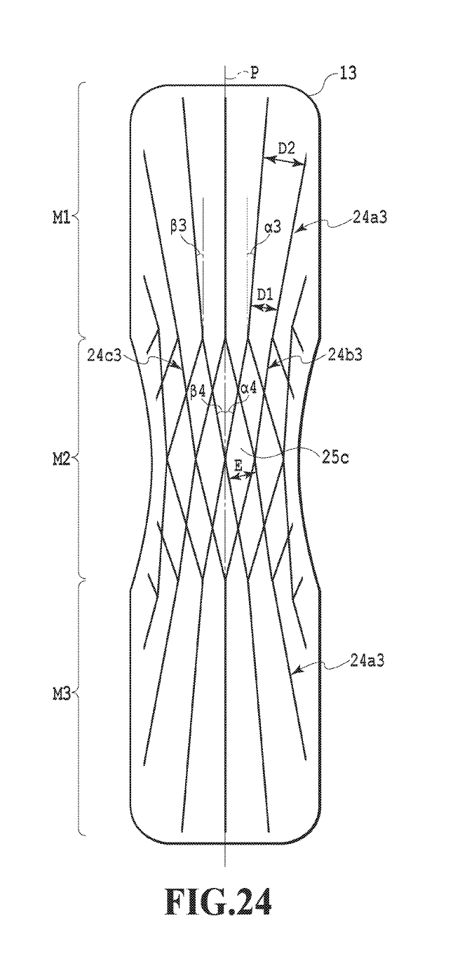

FIG. 24 is a top view seen from the top sheet 14 side, showing an absorber 13 in a disposable diaper 10 according to Embodiment 5 of the present invention. A first difference in shape between the absorber 13 of this embodiment and the absorbers 13 of Embodiments 2 and 3 is that first compressed lines 24a3 are tilted at tilt angles α3 and β3 with respect to the imaginary line P and an interval D between the first compressed lines 24a3 is increased toward the two ends of the absorber 13 in the longitudinal direction. Meanwhile, a second difference is that second compressed lines 24b3 and third compressed lines 24c3 are tilted at tilt angles α4 and β4 smaller than the tilt angles α1 and β1 with respect to the imaginary line P, thereby forming an approximately diamond shape.

Although, as shown in FIG. 24, the tilt angles α3 and 133 of the first compressed lines 24a3 with respect to the imaginary line P are smaller values than the tilt angles α4 and β4 of the second and third compressed lines 24b3 and 24c3 with respect to the imaginary line P, the tilt angles α3 and β3 may be larger than or the same as the tilt angles α4 and β4. Meanwhile, the shape of an absorptive region (uncompressed region) 25c surrounded by the compressed lines 24b3 and 24c3 in the crotch portion M2 is elongated in the longitudinal direction of the absorber 13 compared with the shapes of the absorptive regions 25a and 25b.

Here, the tilt angles α3 and β3 of the first compressed lines 24a3 with respect to the imaginary line P are 6°. The tilt angles α3 and β3 are preferably 2° to 10°. Meanwhile, the tilt angles α4 and β4 of the second and third compressed lines 24b3 and 24c3 with respect to the imaginary line P are 12°. The tilt angles α4 and β4 are preferably 2° to 20°.

In the front body portion M1 and the back body portion M3, an arrangement interval D1 between the first compressed lines 24a3 around the crotch portion M2 is 18 mm and an arrangement interval D2 around the two ends of the absorber 13 in the longitudinal direction is 27 mm. The arrangement interval D (D1 and D2) is preferably 10 mm to 30 mm. Note that the arrangement interval D1 is smaller than the arrangement interval D2 in this embodiment, but may be equal to or larger than the arrangement interval D2.

An arrangement interval E between the second compressed lines 24b3 and between the third compressed lines 24c3 in the crotch portion M2 is 22 mm. The arrangement interval E is preferably 15 mm to 30 mm. Although the tilt angles α3 and β3 are the same value in this embodiment, the two may be different values. Likewise, although the tilt angles α4 and β4 are the same value, the two may be different values. Moreover, although the arrangement intervals E between the second compressed lines 24b3 and between the third compressed lines 24c3 are the same value in this embodiment, the two may be different values.

The absorber 13 of this embodiment can achieve the same effects as those achieved by the absorber 13 of Embodiment 2 as long as the second compressed lines 24b3 and the third compressed lines 24c3 form a lattice pattern and the first compressed lines 24a3 in the front body portion M1 and the back body portion M3 form a linear pattern.

Furthermore, as in the case of Embodiments 2 to 4, the absorber 13 of this embodiment has no compressed lines 24 formed in a peripheral portion thereof with the configuration described above. Thus, the absorber 13 can prevent body fluids from leaking from the leg openings 10L and the waist opening 10W.

The disposable diaper according to this embodiment is applicable to both adults and children. Moreover, in this embodiment, the description is given of, as an example, the pants-type diaper. However, it is needless to say that the disposable diaper according to this embodiment is also applicable to an unfolding-type diaper (so-called tape-type diaper). Furthermore, the absorbent article according to the present invention is not specified as a diaper only, but is applicable to other general absorbent articles such as an absorbent pad.

An absorbent article according to the present invention includes a liquid permeable top surface sheet, a liquid impermeable back surface sheet, and an absorber disposed between the top surface sheet and the back surface sheet. The absorbent article may include a ventilation groove formation region on the top surface sheet side, and the ventilation groove formation region may include ventilation grooves, each having a sidewall and a bottom wall. In the ventilation groove, the top surface sheet and the absorber may be integrally joined together.

In the absorbent article according to the present invention, the ventilation groove formation region may be formed in the center of the absorbent article.

In the absorbent article according to the present invention, the top surface sheet may be fixed to the absorber with an adhesive in the ventilation grooves.

In the absorbent article according to the present invention, the ventilation grooves may be formed intermittently or continuously across the ventilation groove formation region.

In the absorbent article according to the present invention, the ventilation grooves may extend in a direction tilted with respect to an imaginary line from the abdomen side to the dorsal side of a wearer through the crotch when he/she wears the absorbent article.

In the absorbent article according to the present invention, the ventilation grooves include first ventilation grooves extending in a first direction tilted to one side with respect to the imaginary line and second ventilation grooves extending in a second direction tilted to the other side.

In the absorbent article according to the present invention, the first and second ventilation grooves may be tilted within a range of 10 degrees to 80 degrees with respect to the imaginary line.

In the absorbent article according to the present invention, the first or second ventilation grooves which are tilted to the same side and adjacent to each other may have an interval of 10 mm to 100 mm.

In the absorbent article according to the present invention, the ventilation groove formation region does not have to reach two ends of the absorbent article in a width direction perpendicular to the imaginary line.

In the absorbent article according to the present invention, a ratio of the length of the ventilation groove formation region in the width direction may be 30% to 80%.

In the absorbent article according to the present invention, assuming that the time required for a water balloon to move from an upper point to a lower point of a syringe that is placed on the ventilation groove formation region, extends in a vertical direction, and has an inside diameter of 26 mm is a planar air permeability, the planar air permeability may be 3 seconds or less.

The absorbent article according to the present invention may be a disposable diaper in which the absorber has a size that extends across the front body, the crotch, and the back body.

In the absorbent article according to the present invention, the ventilation groove formation region does not have to reach side edges of the front body and side edges of the back body.

An absorbent article according to the present invention includes a liquid permeable top surface sheet, a liquid impermeable back surface sheet, an absorber disposed between the top surface sheet and the back surface sheet. In the absorber, a plurality of recess portions are formed on the top surface sheet side surface. The plurality of recess portions positioned in two end regions on both sides of a center region of the absorber may form a third pattern extending in a direction in which the two end regions facing each other. The plurality of recess portions positioned in the center region of the absorber may form a plurality of first patterns extending in a direction intersecting with the direction in which the two end regions facing each other and a plurality of second patterns extending in a direction intersecting with the first patterns.

In the absorbent article according to the present invention, at east one end of each of the first and second patterns may be communicated with the third pattern.

In the absorbent article according to the present invention, the third patterns may extend parallel to each other.

In the absorbent article according to the present invention, the first pattern and the second pattern may intersect with each other at right angle.

### REFERENCE SIGNS LIST

10 disposable diaper
10F front body region
10R back body region
10C crotch region
10W waist opening
10L leg opening
10J mating portion
10T disposal tape
11 cover sheet
11A pad cover sheet
11B inner cover sheet
11C outer cover sheet
11K, 13A cutout
12 back sheet (back surface sheet)
13 absorber
14 top sheet (top surface sheet)
15 core wrap
16 rubber thread (leg rubber thread)
17 rubber thread (waist rubber thread)
18 side sheet
19 rubber thread (three-dimensional gathering elastic member)
21 ventilation groove
21a first groove
21b second groove
21c bottom
21d sidewall
22 recess portion
23 dent portion
24 compressed line
24a first compressed line
24b second compressed line
24c third compressed line
25 main absorptive region (uncompressed region)
27 urine discharge position
100 measurement device
101 cylinder
102 bottom plate
103 water balloon
L1 longitudinal interval between adjacent recess portions in compressed line
L2 length of recess portion in compressed line
L3 width of recess portion in compressed line
S1, A, C1, C2, D1, D2 formation interval between first compressed lines
S2, S3, B, E formation interval between second compressed lines or third compressed lines

1. An absorbent article comprising:
a liquid permeable top surface sheet;
a liquid impermeable back surface sheet; and
an absorber disposed between the top surface sheet and the back surface sheet, wherein
grooves extend on a top surface sheet side of the absorbent article, and in each of the grooves, a plurality of recess portions formed by integrally joining the top surface sheet and the absorber are arranged, and
each of the recess portions has a sidewall and a bottom.

2. The absorbent article according to claim 1, wherein
the plurality of recess portions are arranged so as to form a plurality of first patterns extending in a direction intersecting with a direction in which two end regions of the absorber on both sides of a center region of the absorber face each other, and form a plurality of second patterns extending in a direction intersecting with the first patterns.

3. The absorbent article according to claim 2, wherein
among the plurality of recess portions, recess portions positioned in the center region of the absorber are arranged so as to form the first patterns and the second patterns, and
recess portions positioned in the two end regions on both sides of the center region of the absorber are arranged so as to form third patterns extending in the direction in which the two end regions facing each other.

4. The absorbent article according to claim 3, wherein
at least one end of each of the first and second patterns is communicated with the third pattern.

5. The absorbent article according to claim 1, wherein
the grooves extend with the recess portions continuously arranged therein.

6. The absorbent article according to claim 1, wherein
the recess portions each include a first recess portion and a second recess portion that is further depressed in the first recess portion.
